(19) United States
(12) Patent Application Publication
(10) Pub. No.: US 2025/0143550 A1
(43) Pub. Date: May 8, 2025

(54) Filter Changing Apparatus for an Endoscopic Camera, Camera Head for an Endoscope, and Retrofit Kit for Retrofitting a Camera Head or an Endoscope

(71) Applicant: KARL STORZ SE & Co KG, Tuttlingen (DE)

(72) Inventors: Kai Kroll, Malsburg-Marzell (DE); Florian Huber, Durchhausen (DE)

(73) Assignee: KARL STORZ SE & Co KG, Tuttlingen (DE)

(21) Appl. No.: 18/940,430

(22) Filed: Nov. 7, 2024

(30) Foreign Application Priority Data
Nov. 7, 2023 (DE) 102023130857.2

(51) Int. Cl. A61B 1/00

(52) U.S. Cl. CPC A61B 1/00186; A61B 1/00096

(57) ABSTRACT

A filter changing apparatus for an endoscopic camera includes base plate, a rotatable filter plate, an optical passage with an optical axis, and at least two pivotable filter holders. The filter plate has a guide groove with a circumferential guide track and is rotatable relative to the base plate. Filter holders are each arranged rotatably on the base plate, and each has a guide element. Each guide element is at least partially arranged in the guide groove, where the guide groove has a first portion, concentric with the optical axis, and a second portion, non-concentric with the optical axis, such that, when at least one guide element is in the concentric portion, a filter holder is arranged in an initial position, free of the optical passage, and/or, when at least one guide element is in the second, non-concentric portion, the associated filter holder can be pivoted into the optical passage.

This application claims priority to German Patent Application No. 102023130062.8 filed Nov. 7, 2023, and entitled “Filterwechselvorrichtung für eine endoskopische Kamera, Kamerakopf für ein Endoskop und Nachrüstsatz zum Nachrüsten eines Kamerakopfes und/oder eines Endoskops,” which is incorporated herein by reference.

The invention relates to a filter changing apparatus for an endoscopic camera, wherein the filter changing apparatus has a base plate, a rotatable filter plate, an optical passage with an optical axis and at least two pivotable filter holders, each with at least one receptacle for an optical filter, wherein the rotatable filter plate has a guide groove with a circumferential guide track and is rotatable relative to the base plate, the filter holders are each arranged rotatably on the base plate by means of a rotation axle and each have a guide element, wherein each guide element is at least partially arranged in the guide groove. Furthermore, the invention relates to a camera head for an endoscope and to a retrofit kit for retrofitting a camera head and/or an endoscope.

In medical and non-medical applications, observation instruments such as endoscopes are used to examine internal cavities of a human or animal body or of an industrial, technical item, such as a pipeline. For imaging, a camera head having an image sensor can be used together with the endoscope. In order to improve the image quality and/or make different observation modes possible, it is known to introduce different filters into the beam path of the observation instrument.

For example, in fluorescence imaging, the item to be examined is exposed to light with an excitation radiation, which excites a fluorophore, which has been previously applied to the item or is already present, to emit light of a certain emission wavelength, wherein the excitation wavelength and the emission wavelength are usually different.

Normally, the emission wavelength is longer than the excitation wavelength. The emitted emission light is usually significantly weaker than other light sources, such as excitation fluorescence light or imaging white light. For these reasons, it is necessary to filter out unwanted wavelength bands by means of a filter so that, if possible, only the desired spectrum and/or the emission wavelength of the fluorophore reaches the camera head in fluorescence mode. For changing between different observation modes, two or more filters are usually introduced one after the other into the beam path of the observation optics, for which purpose various filter changers may be employed.

DE 101 57 057 A1 discloses an apparatus for positioning at least one optical component within an endoscopic system with a housing, through which the optical axis of the endoscopic system runs and in which the at least one component is arranged, which can be pivoted into the beam path and out of it again about a pivot axis running substantially in parallel with a longitudinal axis of the housing, wherein the at least one component, for example a filter for a specific spectral wavelength range, is arranged on a carrier pivotable about the pivot axis. In this case, a smallest distance of an inner wall of the housing from the pivot axis is smaller than a greatest distance of the pivot axis to an outer edge of the at least one component. Due to the closer arrangement of the pivot axis of each pivotable carrier to the inner wall of the housing, the number of carriers for holding as many different filters as possible is limited by the spatially extensive pivoting movement within the limited space of the housing. In addition, the housing is firmly connected to a housing of the optical head of the endoscope. Another disadvantage is that, due to the spatially closer arrangement of the pivot axis to the inner wall of the housing, a large number of movable individual parts is required in order to introduce a plurality of filters into the beam path. This results in increased costs and installation effort. In addition, this increases the risk of wear, inaccuracies in the particular pivoting movement, and, consequently, malfunctions.

From the Applicant's own prior art (German application number 10 2022 131 502.9), a filter changing apparatus for an endoscopic camera head with at least three optical filters is known. The filter changing apparatus has a groove with a non-circular guide track and a rotating element, where, by rotating the rotating element, carriers arranged one after the other in the groove are moved into and out of the beam path by means of a guide element partially arranged in the groove, due to an at least partial movement along the non-circular guide track. The disadvantage here is that, for arranging a plurality of filters, a carrier arm each with a guide element is required so that, due to the larger number of guide elements or filter carriers arranged directly in the groove, wear and/or disruptive influences on the function of the filter changing apparatus can occur due to the frictional forces that occur. In addition, the pivotable carrier arms overlap so that a larger installation space is required along the optical axis.

In order to improve upon the prior art a filter changing apparatus for an endoscopic camera is disclosed, wherein the filter changing apparatus has a base plate, a rotatable filter plate, an optical passage with an optical axis and at least two pivotable filter holders, each with at least one receptacle for an optical filter, wherein the rotatable filter plate has a guide groove with a circumferential guide track and is rotatable relative to the base plate, the filter holders are each arranged rotatably on the base plate by means of a rotation axle and each have a guide element, wherein each guide element is at least partially arranged in the guide groove and the guide groove has a first portion, concentric with the optical axis, and a second portion, non-concentric with the optical axis, so that, when at least one guide element is arranged in the concentric portion, the particular filter holder is arranged in an initial position, free of the optical passage, and/or, when at least one guide element is arranged in the second, non-concentric portion, the associated filter holder can be pivoted into the optical passage.

This provides a filter changing apparatus where two or more filters can be precisely and efficiently pivoted into and out of the beam path of an endoscopic camera.

It is particularly advantageous that the geometry and the course of the guide groove with a first, concentric portion and a second, non-concentric portion define and predetermine a pivoted-out position, pivoted-in position, intermediate position, initial position, and/or rest position of each filter holder, whereby the particular pivotable filter holder or the pivotable filter holders can be used precisely and efficiently. In addition to determining a defined alignment and/or position of each filter holder to the optical axis and/or the passage due to the geometry and the course of the guide groove and the positioning of the particular guide element in the guide groove, the guide element is held in the guide groove so that, even in the event of wear and thus play of the rotation axle of the particular filter holder, the predefined pivoted-out position, pivoted-in position, intermediate position, initial position and/or rest position of the particular filter holder is still maintained. Consequently, although a pivoting movement of the particular filter holder is initiated via the rotation axle, the precise maintenance of the desired position of the particular filter holder is predetermined by the interaction of the guide track with the guide element of the filter holder, thus ensuring that the desired position is precisely assumed and/or maintained. Thus, depending on the position of the particular guide element in the first, concentric portion or the second, non-concentric portion, defined, successive local positions (also called guide groove positions) are predetermined for the particular guide element in the guide groove due to the design, shape and course of these two portions, at which positions the associated filter holder maintains and/or assumes a pivoted-out position, pivoted-in position, intermediate position, initial position and/or rest position and is in a particular spatial alignment with the optical passage. The order and timing of the filter changes are thus predetermined by the course of the guide groove.

As a result, when the particular guide element moving in the guide groove reaches a predetermined local position and/or guide groove position of the second, non-concentric portion, a pivoting-in or pivoting-out movement of the particular filter holder is or will be automatically realized.

Since the guide groove is circumferential all around and, consequently, the end of the first, concentric portion transitions into the beginning of the non-concentric portion and the end of the non-concentric portion transitions into the beginning of the first, concentric portion, the particular guide element can circulate all around in the guide groove without limitation and the rotatable filter plate can consequently be constantly rotated by the user.

In addition, the course of the guide groove with the first, concentric portion and the second, non-concentric portion, due to their shape and the thereby predetermined defined positions, at which the associated filter holder remains in the initial position or performs a certain predetermined movement, makes it possible that the positions and movements of the at least two pivotable filter holders are optimally coordinated in time and space. This makes it possible for the at least two filter holders to be arranged in one plane and/or next to one another. Consequently, the coordinated spatial arrangements and movements of the filter holders and the compact design of the filter holders themselves make a very small design of the filter changing apparatus possible. Above all, the filter changing apparatus has a small dimension along the optical axis due to the arrangement of the at least two filter holders in one plane.

In addition to the shape and the course of the guide groove with the first, concentric portion and the second, non-concentric portion and thus the particular position of each guide element within the guide groove, the at least two filter holders are arranged on the base plate by means of their particular rotation axles at a predetermined spatial distance and in a spatial position relative to one another. The spatial arrangement of the first guide element and of the second guide element and their distance along the guide track are thereby predetermined. If, for example, the first guide element is located within the first, concentric portion and the first filter holder is thus in the initial position, free of the passage, the second guide element is arranged within the second, non-concentric portion and, depending on the local position along the course of the second, non-concentric portion, performs a corresponding movement or assumes an intermediate position or initial position.

An essential idea of the invention is based on forming a guide groove in a rotatable filter plate with a circumferential guide track specifically with a first portion, concentric with the optical axis, and a second portion, non-concentric with the optical axis, wherein, due to the particular shape and the course of the concentric and non-concentric portions, a guide element of a particular filter holder is moved in and along the guide groove when the filter plate is rotated, whereby, when the guide element reaches a predetermined position and/or guide groove position in the concentric or the non-concentric portion, an initial position, intermediate position and/or rest position or a specific movement for pivoting into or out of the optical passage is imposed on the associated filter holder. The course of the concentric portion and of the non-concentric portion predetermines different, successive guide groove positions for the guide elements and, as a result, a spatial alignment of the particular filter holder to the optical passage. In interaction with the rotatable arrangement of the at least two filter holders on the base plate by means of a particular rotation axle, a timing and an order of changing the filters is predetermined by the particular guide groove position of the guide element in the guide groove. This provides a filter changing apparatus by means of which different filters, in particular fluorescence filters, can be pivoted into and out of the beam path of an endoscopic camera precisely, quickly, efficiently and with a long service life.

The following terminology is explained:

A “filter changing apparatus” is in particular an apparatus by means of which one of two or more filters can be moved into and out of the optical beam path. The filter changing apparatus can in particular be activated manually or automatically to change the filters by rotating the rotatable filter plate. In the process, changing between two filter receptacles and/or filters of a single filter holder or between two filter receptacles and/or filters of two filter holders can take place. The filter changing apparatus in particular has at least two optical filters, preferably at least three optical filters. Optionally, the filter changing apparatus can also have a filter receptacle which does not have an optical filter and thus allows free passage through the optical beam path. The filter changing apparatus can thus also introduce an empty filter receptacle into the optical passage and into the beam path. Likewise, instead of omitting an optical filter, free passage can also be made possible by a non-filtering optical element, such as a glass pane. A glass pane used as a window can also have an anti-reflective coating. The filter changing apparatus can in particular be integrated in a camera or can be connectable as a separate apparatus, for example designed as a snap-on filter, to the camera and/or an endoscope.

An “optical filter” (also simply referred to as “filter”) is in particular an optical element which selects the incident radiation and/or incident beams on the basis of specific properties, such as a wavelength, a polarization state, an angle of incidence and/or a direction of incidence, and thus allows them to pass through or prevents them from passing through. Likewise, an optical filter can change the properties of the light passing through it, for example by converting circularly polarized light into linearly polarized light. In particular, an optical filter can block a specific spectral wavelength band. An optical filter may, for example, be a graduated filter, an edge filter, a polarizing filter or an interference filter. An interference filter in particular has a coating which blocks light of a certain spectral range or allows it to pass through. The optical filter can in particular be used as an observation filter and/or detection filter, fluorescence observation filter or excitation filter. A fluorescence observation filter (also referred to as a fluorescence filter) is in particular an optical polychroic interference filter for separating the emitted fluorescence light from the excitation light used. The fluorescence filter thus blocks the specific fluorescence excitation radiation and allows the fluorescence emission radiation to pass along the optical beam path. Preferably, the fluorescence filter completely blocks the excitation light while allowing the fluorescence emission light to pass through, which usually has a longer wavelength than the excitation light. The optical filter in particular comprises glass or a crystalline material. The optical filter may be planar or designed as a filter lens. In principle, instead of the optical filter, another optical element, such as a lens, an aperture, a polarizer or a similar optical element, can also be arranged in the filter changing apparatus and/or the filter receptacle.

An “optical passage” is in particular a hollow space in the filter changing apparatus through which light can pass. An optical passage is in particular a continuous opening through the rotatable filter plate, further constituents and/or the housing of the filter changing apparatus. The optical passage is in particular arranged around the center of the cross section of the rotatable filter plate and/or around the optical axis. The optical passage extends in particular along the optical axis. In particular, a filter receptacle and/or an optical filter can be arranged in front of and/or in the optical passage in the light propagation direction. Likewise, when light passes through, the optical passage may be free of an arranged optical filter and/or a receptacle. In principle, the optical passage can have any cross-sectional shape, but the optical passage is preferably circular in cross section.

An “optical axis” is in particular a line along which a degree of rotational symmetry exists in an optical system. The optical axis is in particular an imaginary line that defines a path along which light propagates through the filter changing apparatus and/or the camera toward an image sensor. Preferably, the optical axis runs through the curvature means of the particular pivoted-in filter and/or of a downstream lens system and/or objective system. However, the optical axis can also be bent and/or directed by a lens, an optical element and/or one of the optical filters. The optical beam path as a geometric course of light beams is in particular arranged in and/or around the optical axis and runs along, converges toward and/or disperses from the optical axis.

A “rotatable filter plate” is in particular a plate which is rotatable and has a circumferential guide groove. The filter plate is in particular rotatable about its point of rotation and/or its axis of rotation. The filter plate is in particular rotatable relative to a base plate of the filter changing apparatus. The filter plate can in particular be driven and rotated by hand and/or by means of a drive unit and/or a motor. For this purpose, the outer peripheral surface may, for example, be driven as a contact surface by a drive unit and/or a gearing mechanism acting at and/or on this contact surface. Accordingly, the contact surface can be specifically designed for driving, for example can have external gears. The filter plate is in particular a flat, planar component, with its opposite plate surfaces aligned substantially perpendicularly to the optical axis. The filter plate in particular has an optical passage through its material thickness at the center of the plate surface. In order to keep the size of the filter changing apparatus along the optical axis as small as possible, the filter plate in particular has a low material thickness. In principle, it should be pointed out that, instead of a filter plate, a rod-shaped component, such as a cylinder with a cylinder axis aligned along the optical axis, can also be arranged. The guide groove of the filter plate can be introduced into one of the two opposite plate surfaces and thus have a smaller groove depth than the material thickness of the filter plate, or the guide groove passes completely through the material thickness of the filter plate. By rotating the filter plate, the particular guide element arranged in the guide groove moves in and along the guide groove, which induces a rotational movement of the particular filter holder about its rotation axle. Depending on the movement and/or arrangement of the particular guide element in the first, concentric portion, the filter holder is arranged in an initial position, free of the optical passage, or, when the particular guide element is moved and/or arranged in the second, non-concentric portion, the associated filter holder is pivoted into or out of the optical passage or is in an intermediate position. Basically, the rotatable filter plate can be rotated clockwise and counterclockwise. Due to the design of the guide groove with a closed, circumferential guide track, the rotatable filter plate is infinite and can thus be rotated with any number of revolutions in either of the two directions of rotation. In order to reduce the frictional forces between the walls of the guide groove and the outer surface of each guide element, the rotatable filter plate in particular comprises a material with a low coefficient of friction, for example aluminum or a polymeric material such as PTFE. Depending on the shape and/or course of the guide groove at a position and/or a subportion in which the particular guide element is arranged and/or moves, when the filter plate rotates, one of the two inner walls or both inner walls of the guide groove press against the outer surface of the particular guide element and thereby convert the rotational movement of the filter plate into a further movement of the particular guide element in the guide groove. Since the filter holder of the particular guide element is connected not only indirectly in the guide groove via the guide element but also rotatably to the stationary base plate by means of its rotation axle, the pivoting movement of the filter holder and its direction depends on the shape and course of the guide groove in the region in which the particular guide element is located.

A “guide groove” is in particular a slot and/or a cut in a material and/or in a surface of the filter plate. A guide groove is in particular an elongated, circumferentially all-round depression in the filter plate or an elongated, circumferentially all-round cut through the filter plate. The guide groove in particular has at least two differently designed portions in its longitudinal direction. In the first portion concentric with the optical axis, the guide groove has the shape of a circular arc. In this first portion concentric with the optical axis, the guide groove has a constant distance to the optical axis. Here, the optical axis is also the center of the guide groove, which is designed as a circular arc, in this first, concentric portion. A distance and a radius from the optical axis to the circular-arc-shaped guide groove in the first, concentric portion is thus constant. Consequently, a guide element arranged in this first portion concentric with the optical axis is moved uniformly in this portion of the guide groove due to a rotation of the filter plate, wherein the two opposite inner walls of the guide groove act uniformly on the guide element so that the filter holder is not forced to rotate about its rotation axle. Accordingly, as long as the guide element is arranged in this first portion concentric with the optical axis, the filter holder remains in an initial position and/or rest position, free of the optical passage. This filter holder therefore does not move relative to the base plate. In order to provide a circumferential guide track, a first end and a second end of the first, concentric portion are connected to the corresponding two ends of the second portion, non-concentric with the optical axis, so that the at least two guide elements arranged in the guide groove can move continuously, circumferentially all around in the guide groove when the filter plate rotates.

A second portion of the guide groove which is non-concentric with the optical axis is in particular understood to mean that the guide groove in this portion does not run at the same distance to the optical axis as the center. In particular, the second, non-concentric portion does not have a circular arc shape along its entire course but is irregular and formed with different distances and subportions to the optical axis. Only at its two ends does the second portion, non-concentric with the optical axis, have the same distance to the optical axis as the first, concentric portion in order to make possible a direct and smooth transition from the second, non-concentric portion to the first, concentric portion and vice versa during the movement of the particular guide element. Nevertheless, the second, non-concentric portion may have the shape of two or more circular arcs following one another and/or arranged in a row. However, these circular ares in particular do not have the optical axis as their center. In addition, the centers of the circular arc portions of the second, non-concentric portion can each lie within the circumferential guide groove and thus between the guide groove and the optical axis or outside and thus between the guide groove and the outer circumference of the filter plate. Consequently, the second, non-concentric portion of the guide groove can have circular-arc-shaped subportions which are arranged in a row and are closer to the optical axis and/or the free passage than the first, concentric portion and/or are at a greater distance to the optical axis and/or the free passage than the first, concentric portion. In this case, the maximum outer diameter of each circular arc segment can be aligned toward the optical axis and/or the free passage or toward the outer diameter of the filter plate. The successive segment-arc-shaped subportions of the second, non-concentric portion may also have different segment heights, different radii, center angles and/or circular arc lengths. The non-concentric portion may also have at least one linear subportion.

The guide groove, its concentric portion and/or its non-concentric portion in particular have one guide groove position or a plurality of guide groove positions. A “guide groove position” is in particular a local position in the guide groove and/or the particular portion at which, due to the rotation of the filter plate, the inner walls of the guide groove impose a pressure and/or a change in the direction of movement on the guide element moving into or present in the guide groove position, in such a way that the associated pivotable filter holder remains in its spatial alignment or a pivoting movement is imposed on it. A guide groove position may, for example, be a neutral transition position or intermediate position of the second, non-concentric portion or a starting position or an end position of the first, concentric portion.

After passing through the first, concentric portion in a clockwise direction, the second, non-concentric portion at the transition can have a neutral transition position with a circular-arc-shaped segment, which has a center in the region internally enclosed by the guide groove, so that this subportion in its further course approaches the optical axis until the second, non-concentric portion has reached a minimum distance to the optical axis and/or the free passage in an intermediate position at a minimum distance. Here, the intermediate position at a minimum distance is in particular defined by a short circular arc segment, which is oppositely aligned with a center outside the guide groove and thus is formed between the guide groove and the outer circumference of the filter plate. When this intermediate position at a minimum distance is reached, due to the forces acting on the guide element there, a filter receptacle of the associated filter holder pivots in.

Starting from this intermediate position at a minimum distance, the further course of the guide groove can bend outward again with a larger radius so that the center of this subportion again lies within the region enclosed by the guide groove. In particular, the course of the second, non-concentric portion intersects the radius of the first, concentric portion in a neutral transition position and subsequently leads even further away to a greater distance to the optical axis until a guide groove position is reached in which there is an outermost plateau with a greatest distance to the optical axis. Subsequently, the second, non-concentric portion in particular runs inward again in a further subportion until it reaches the radius of the first, concentric portion and transitions into it.

In principle, it should be pointed out that the terms “first” and “second” portion, filter arm, filter and other terms are only used for differentiation. For example, which portion is passed through first and which portion is passed through second when moving a particular guide element in the guide groove depends on the clockwise or counterclockwise direction of rotation.

The asymmetrical shape of the second, non-concentric portion, in particular by arranging different circular arc segments, differently shaped subportions, guide groove positions and/or locking positions, specifically predetermines a particular movement of the guide element and thus of the pivotable filter holder.

The term “initial position” (also called “rest position”) is in particular understood to mean a maintained position and/or alignment of the particular filter holder, free of the optical passage. In this case, the associated guide element is in particular arranged in the guide groove in the first portion, concentric with the optical axis, and/or on the radius thereof. When the particular guide element moves in the first portion concentric with the optical axis, the associated filter holder does not perform any rotational and/or pivoting movement about its rotation axle. In the initial position, the associated filter holder is arranged in the longitudinal direction with its outer side facing inward, in particular at a distance next to the outer diameter of the optical passage. In the rest position, the particular filter holder is thus in particular aligned horizontally and located below or above the optical passage. The initial position can also be present in the non-concentric portion of the guide groove where the corresponding subportion intersects the radius of the concentric portion at a predetermined guide groove position. If a guide element is located at this position, which is in particular also referred to as the neutral intermediate position or transition position, the associated filter holder is pivoted out of the optical passage.

A “filter holder” is in particular an elongated element and/or an arm, which is arranged on the base plate so as to be rotatable about its rotation axle. The filter holder in particular has a first bore, in which the rotation axle is arranged. This first bore may be partially or completely continuous through a material thickness of the filter holder transverse to its longitudinal direction. An opening of this first bore is in particular aligned toward the base plate. The rotation axle of the particular filter holder is in particular arranged in the center of the filter holder. The rotation axle may, for example, be designed as a shaft. Furthermore, the filter holder in particular has on its underside a second bore, in which the guide element is received and/or fastened. The underside of the filter holder is in particular the side and/or surface that is facing the guide groove and/or the rotatable filter plate. The filter holder has at least one receptacle for an optical filter. Preferably, each filter holder has two receptacles at its opposite ends.

A “receptacle” (also called a “filter receptacle”) is in particular a hollow body with a partial or complete external enclosure and/or border, into which hollow body an optical filter can be inserted and which hollow body at least partially encloses the optical filter at its circumference. A receptacle may, for example, be a short tubular body. The receptacle in particular forms a protective casing for the optical filter.

A “guide element” is in particular an element that has an at least slightly smaller outer diameter than the inner diameter of the guide groove so that the guide element is at least partially arranged in and/or within the guide groove. The guide element is in particular firmly or detachably connected to the associated filter holder. The guide element can in particular be connected directly to the filter holder or indirectly to the filter holder via a connecting element and/or a ball bearing. The term “at least partially arranged in the guide groove” is in particular understood to mean that the length of the guide element arranged in the guide groove does not necessarily have to extend over an entire depth of the guide groove, and thus the cavity of the guide groove, substantially orthogonally to the surface of the filter plate.

A “base plate” is in particular a component of the filter changing apparatus, on and/or to which the at least two pivotable filter holders are rotatably fastened. The base plate is in particular free of a groove. A base plate may also be a housing part and/or a housing cover of the filter changing apparatus.

A “camera” (also called a “camera head”) is in particular a piece of equipment for receiving image light along an optical axis from an endoscope and for focusing the received image light on at least one image sensor. In addition to the at least one image sensor, the camera may in particular have an aperture or a window for letting through the received image light and a lens system for focusing the image light on the at least one image sensor. The image data recorded by at least one image sensor can in particular be transmitted electronically by the camera head to a display system and/or to an image processing unit in order to display the endoscopic image to the user. The camera may have means for recognizing the connected endoscope and for processing algorithms. A connector for connecting an endoscope to the camera can be arranged at the distal end of the endoscope and/or the proximal end of the camera head. The filter changing apparatus according to the invention itself may also be designed as a connector for connecting an endoscope to a camera.

An “endoscope” is in particular a medical or industrial piece of equipment for endoscopic examination and inspection of a human or animal body cavity and/or an industrial cavity, such as a tube. The endoscope in particular has a handpiece, a shaft, a light source, a light guide, a sensor and/or a camera. The endoscope is in particular a video endoscope, which has digital image recording and image transmission and thus an integrated or connectable camera. In addition to medical and veterinary applications, an endoscope and/or video endoscope may, however, also be used for industrial purposes, for example for visual inspection in hard-to-reach cavities. In industrial applications, an endoscope is often referred to as a borescope.

An “image sensor” is in particular a light-sensitive electronic component which is based on an internal photoelectric effect. By means of the image sensor, one or more images from the viewing area of the imaging apparatus are in particular recorded and converted into electronic signals. The image sensor has a sensor plane in the image plane of the optical system, of a lens system and/or of the objective. An electronic image sensor may in particular be a CCD sensor (charge-coupled device) or a CMOS sensor (complementary metal-oxide semiconductor).

In a further embodiment of the filter changing apparatus, the first, concentric portion of the guide groove has a length with an angular width, with the optical axis as the angle vertex, in a range of 170° to 190°, in particular of 175° to 185°, preferably of 178° to 182°.

Since the angular width and thus the central angle on the optical axis is slightly less or slightly more than 180° or most preferably exactly 180°, at least one of the at least two filter holders is in the initial position during a semicircular rotation.

To ensure that both filter holders are arranged in a pivoted-out alignment, free of the optical passage, and/or that, between each pivoted-in position when rotating the filter plate, there is a subsequent guide groove position in which no filter and/or no receptacle is pivoted into the optical passage, the first, concentric portion of the guide groove can have a starting position at its one end and an end position at its other end so that, when the guide element of the first filter holder is arranged in the starting position and the guide element of the second filter holder is arranged in the end position, both filter holders are in a position, free of the optical passage, and/or in the particular initial position.

Since, after each pivoting-in of the receptacle of a filter holder into the optical passage, a position without pivoting a receptacle and/or a filter of the corresponding filter holder into the beam path is subsequently reached when the filter plate is rotated further, the user of the filter changing apparatus is clearly shown that a change is taking place between the filter already used and the filter to be used next. Specifically keeping the optical passage clear in this way between the pivoted-in filters prevents the filters from being confused and a wrong filter from being applied unintentionally, for example when using different fluorescence filters.

In a further embodiment of the filter changing apparatus, the rotation axle of each filter holder is arranged between the optical axis and the guide groove or between the guide groove and an outer diameter of the rotatable filter plate.

Since the rotation axle of each filter holder is firmly connected to the base plate so that the filter holder is rotatably mounted, the arrangement of the rotation axles in the internal region enclosed by the guide groove and/or in the region around the outside of the guide groove can be used to determine the rotation radii of the filter holders and/or to adjust an overlap of the rotation radii of the filter holders during pivoting-in and/or pivoting-out in relation to the geometry and the course of the guide groove. By specifically placing the rotation axles of the filter holders above the surface of the base plate, it is also possible, with an adapted course of the guide groove with its two portions, to arrange more than two filter holders in the filter changing apparatus.

In order to increase the number of changeable filters, the filter holder(s) can have a receptacle at each of two opposite ends and the rotation axle can be arranged centrally between the two opposite ends.

When two filter holders are arranged in the filter changing apparatus, it is thus possible to pivot in four receptacles and/or filters one after the other. In addition, the frictional forces are reduced and potential wear is thus decreased since only one rotation axle of the filter holder and also only one guide element in the guide groove are necessary for two receptacles and/or filters.

In this case, it is particularly advantageous that the two filters of a filter holder can first be pivoted one after the other into the optical passage and thus into the beam path before a change to a second filter holder takes place. This means that when changing between the two filters of one filter holder, filter change times are shorter since the filter holder only has to rotate counterclockwise or clockwise about its rotation axle in order to change from its first filter to its second filter or vice versa.

In a further embodiment of the filter changing apparatus, the guide element of each filter holder is arranged between the rotation axle and one of the two receptacles, in particular closer to the rotation axle than to the particular receptacle.

By positioning the guide element in relation to the rotation axle on the particular filter holder, taking into account the course of the guide groove in which the guide element moves when the filter plate rotates, a defined rotation of the filter holder and thus a pivoting-in and pivoting-out of the particular receptacle into and out of the optical passage can be realized.

In order to provide a defined pivoted-in position for a receptacle of a filter holder in the optical passage, the second, non-concentric portion of the guide groove can have a first subportion with a guide groove position at a maximum distance, which subportion has a greater distance to the optical axis than the first, concentric portion so that, when the particular guide element is arranged at the guide groove position at a maximum distance, the particular filter holder can be pivoted into the optical passage.

In this case, the rotation axle of the second filter holder can be arranged on the base plate in such a way that the associated guide element is located in the first, concentric portion of the guide groove so that this filter holder is arranged in the initial position, free of the optical passage, while the first filter holder is pivoted in due to the arrangement of the first guide element at the guide groove position at a maximum distance.

In a further embodiment of the filter changing apparatus, the second, non-concentric portion of the guide groove has a second subportion with a guide groove position at a minimum distance, which subportion has a smaller distance to the optical axis than the first, concentric portion so that, when the particular guide element is arranged at the guide groove position at a minimum distance, the receptacle of the particular filter holder that is arranged closest to the guide element is pivoted into the optical passage.

As a result, if the filter holder whose guide element is located in the second, non-concentric portion of the guide groove has two receptacles, both receptacles of this filter holder can be pivoted into the optical passage one after the other when this guide element passes through this second, non-concentric portion.

In order to realize a clear temporal and user-specific separation between the pivoting-in of a receptacle and/or a filter at the guide groove position at a maximum distance and the pivoting-in of the second receptacle and/or the second filter at the guide groove position at a minimum distance, a transition portion with a transition position can be arranged between the first subportion and the second subportion of the second, non-concentric portion so that, when the particular guide element is arranged, both receptacles of the particular filter holder are pivoted out of the optical passage.

This clearly shows the user of the filter changing apparatus that a change of the filters has taken place and consequently prevents confusion of two consecutive filters due to the intermediate position free of a filter in the optical passage.

In a further embodiment of the filter changing apparatus, the transition position and the first, concentric portion are at the same distance to the optical axis so that, when the particular guide element is moved along the guide groove, the particular filter holder can be pivoted out directly into a position free of the optical passage and/or into the initial position after being arranged in the optical passage.

Since the transition portion at the transition position has the same distance from the optical axis as the first, concentric portion due to a change from the guide groove position at a maximum distance to the guide groove position at a minimum distance (or exactly the other way around if the direction of rotation of the filter plate is reversed), the two guide elements each lie on the radius of the first, concentric portion around the optical axis so that, in this case, all receptacles and filters are pivoted out and both filter holders are in the initial position.

In order to make a compact arrangement of the filter holders and/or a small diameter of the filter changing apparatus transverse to the optical axis possible, the filter holder(s) can be dumbbell-shaped or double-lobe-shaped.

The geometric shape of a dumbbell or double lobe of each filter holder makes it possible for one filter holder to be pivoted out of the optical passage while the other filter holder is pivoted into the optical passage at the same time, without the two filter holders colliding despite overlapping rotation radii. Due to the shape of the filter holders, a compact arrangement of the filter holders and simultaneous pivoting movements of both filter holders are thus possible, as a result of which the diameter of the filter plate, the base plate and/or the entire filter changing apparatus can be correspondingly small. In this case, a receptacle and/or a filter can be arranged in each of the two opposite ends of the dumbbell-shaped or double-lobe-shaped filter holder. Due to the two dumbbell-shaped and/or double-lobe-shaped filter holders, the overlapping rotation radii can have an overlap region which, in its smallest dimension, corresponds at least to the outer diameter of the particular filter holder in the region of the filter receptacle.

In a further embodiment, the filter changing apparatus has a feedback device when a pivoted-in position and/or pivoted-out position is reached.

The feedback device can be, for example, configured in such a way that, when a guide groove position, a pivoted-in position, and/or a pivoted-out position of the filter holder is reached, the user is given tactile and/or haptic feedback via the rotatable filter plate, which is operated from the outside by the user. For this purpose, the feedback device may have, for example, one or more pressure pieces, which cause firm engagement in each guide groove position, pivoted-in position, and/or pivoted-out position. The pressure piece(s) can be arranged like the rotation axles of the filter holders on the base plate and/or a housing cover. Likewise, the feedback device may have, for example, spring-loaded balls in mini bores in the base plate to provide haptic feedback to the user. Of course, the feedback device may also emit an auditory or optical feedback signal to the user instead of or in addition to the haptic and/or tactile feedback.

In order to make both clockwise rotation and counterclockwise rotation of each filter holder possible and to arrange the filter holders in a space-saving manner on and/or at the base plate, the first, concentric portion and the second, non-concentric portion are designed in such a way that the filter holders have opposite directions of rotation about the particular rotation axle and/or have overlapping rotation radii.

For this purpose, the guide groove can be designed such that, when the particular guide element is positioned at a greater distance than the distance of the first, concentric portion from the optical axis and/or at the guide groove position at a maximum distance, the associated filter holder rotates clockwise about its rotation axle. In the process, the guide element is pressed externally over the rotation axle and is at a greater distance to the optical axis and/or further outward than the rotation axle. Accordingly, when the particular guide element reaches a position with a smaller distance than the distance of the first, concentric portion to the optical axis and/or the guide groove position at a minimum distance, the associated filter holder rotates counterclockwise, wherein the guide element is pressed internally under the rotation axle and is at a smaller distance to the optical axis and/or further inward than the rotation axle.

In a further aspect of the invention, the object is achieved by a camera head for an endoscope, wherein the camera head has an image sensor, an opening for receiving light of an image along an optical path, and an optical lens system for focusing the light on the image sensor, wherein the camera head has at least one filter changing apparatus as described above.

This provides a camera head on or in which a compact, space-saving filter changing apparatus is arranged. If at least one filter changing apparatus is arranged directly in the camera head, the camera head can be detachably connected to different types of endoscopes. Of course, the camera head can also have two or more filter changing apparatuses in series in an optical path and/or along the optical axis.

By stacking two or more filter changing apparatuses in series either within the camera head or alternatively between the proximal end of an endoscope and the distal end of the camera head, different possible filter settings and applications can be realized, in particular in multispectral imaging and/or in a broad application of different fluorophores in fluorescence imaging.

In order to adjust a currently used filter configuration or adapt it to desired different observation modes, the camera head may comprise a detection unit for detecting an identification of each optical filter in the optical path. Likewise, the camera head can have a control unit for adjusting the rotation speed of the filter plate and for checking and/or adjusting the particular optical filter, arranged in the beam path, according to the operating mode selected at the time.

In a further aspect of the invention, the object is achieved by a retrofit kit for retrofitting a camera head and/or an endoscope, wherein the retrofit kit has at least one filter changing apparatus as described above, so that the filter changing apparatus can be arranged between a proximal end of the endoscope and a distal end of the camera head.

This provides a retrofit kit (also referred to as an “adapter”) with at least one filter changing apparatus, which retrofit kit simultaneously serves both as a connector between an existing endoscope and an existing camera head and to make different observation modes possible. In addition, the retrofit kit may also comprise two or more filter changing apparatuses arranged in series between the endoscope and the camera head, or one filter changing apparatus may be replaced by another filter changing apparatus to make different applications and/or observation options possible.

The invention is explained in more detail below with reference to exemplary embodiments, and in particular with respect to the figures described below.

FIG. 1 is a schematic three-dimensional detail view of an endoscope system with an endoscope, a filter changer, and a camera head.

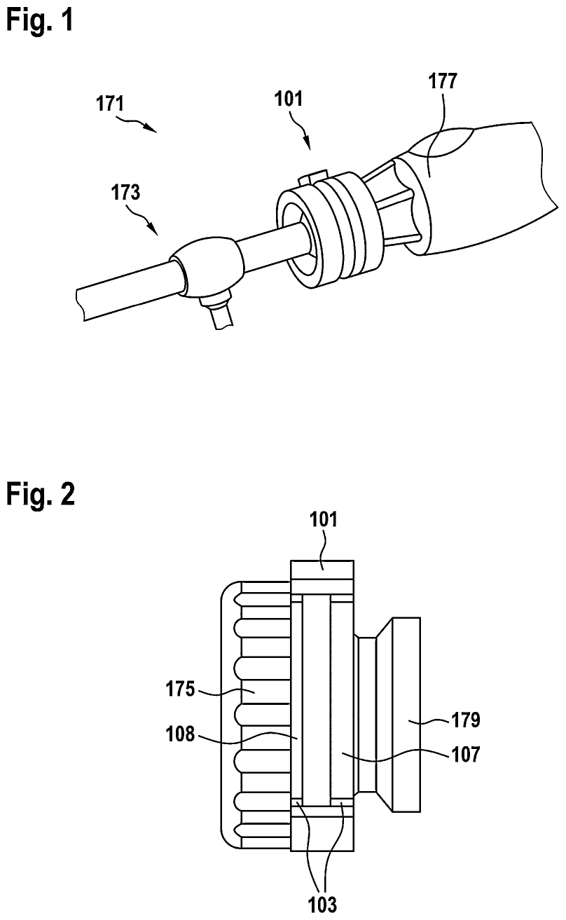

FIG. 2 is a three-dimensional representation of a filter changer in side view with an endoscope receptacle and a camera receptacle.

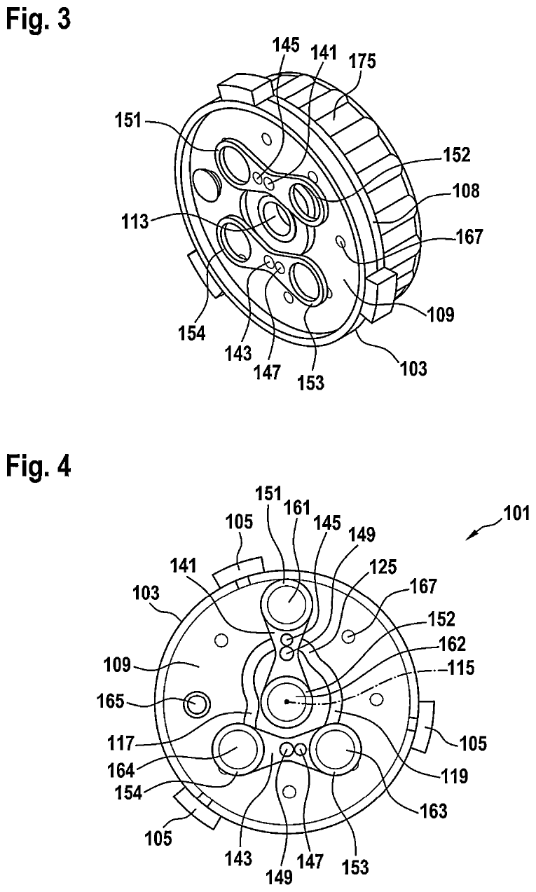

FIG. 3 is a three-dimensional representation of the filter changer of FIG. 2 with two filter pivot arms in an initial position in a sectional view.

FIG. 4 is a schematic representation of the filter changer of FIG. 3 with the housing open and a filter pivot arm pivoted in.

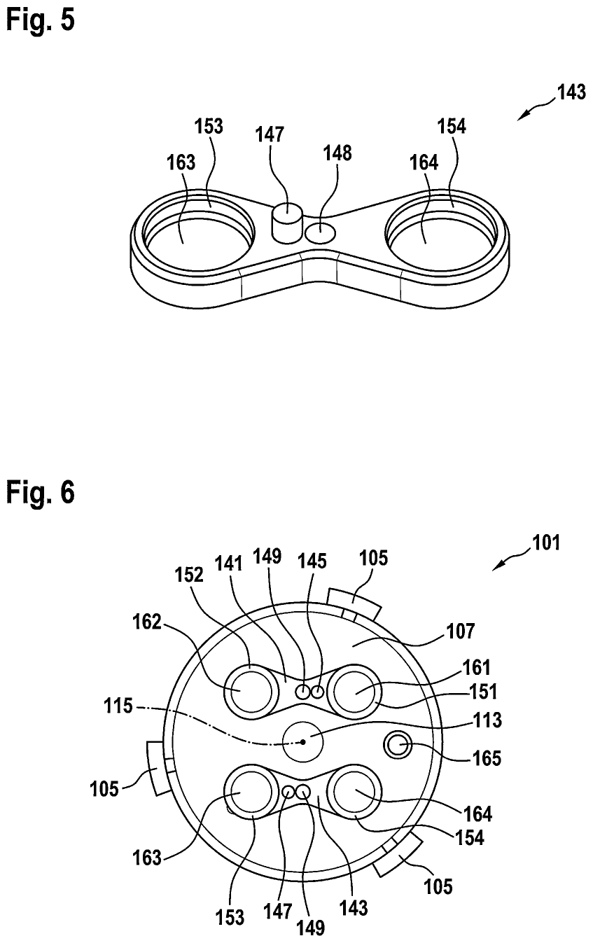

FIG. 5 is a three-dimensional representation of a filter pivot arm.

FIG. 6 is a schematic three-dimensional representation of a cover of the filter changer with the filter pivot arms arranged thereon.

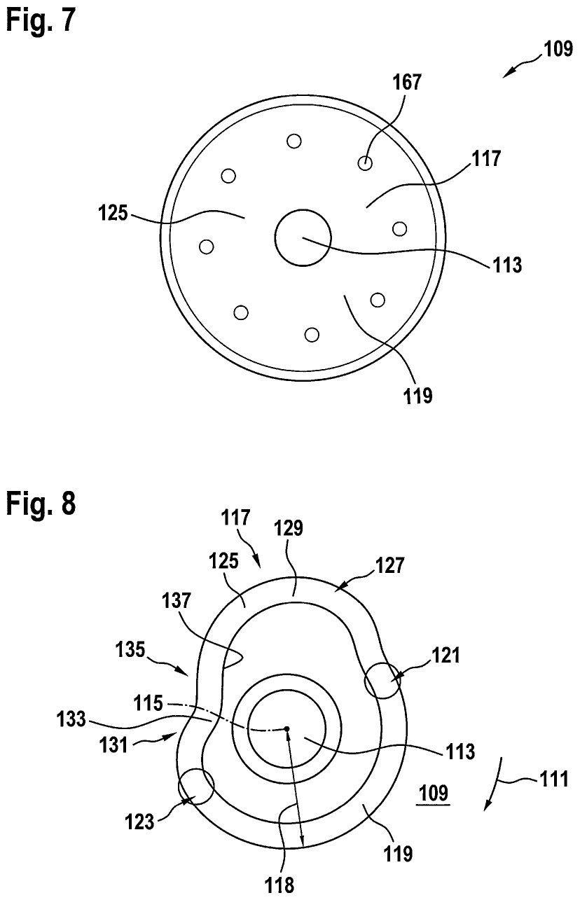

FIG. 7 is a schematic representation of a filter wheel with a circumferential guide groove.

FIG. 8 is a schematic representation of the filter wheel with the guide groove with subportions and guide groove positions.

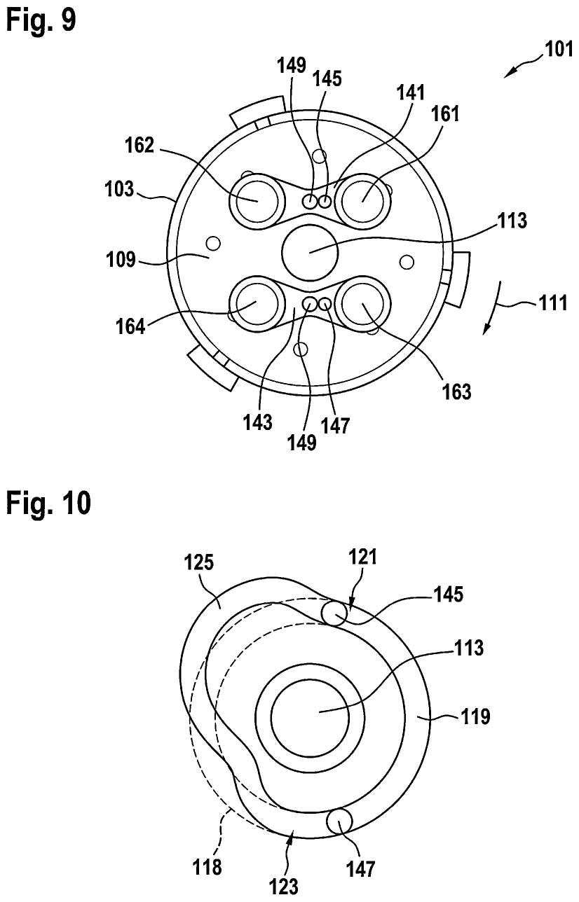

FIG. 9 is a schematic representation of the filter changer with both filter pivot arms in the initial position.

FIG. 10 is a schematic representation of the guide groove corresponding to FIG. 9.

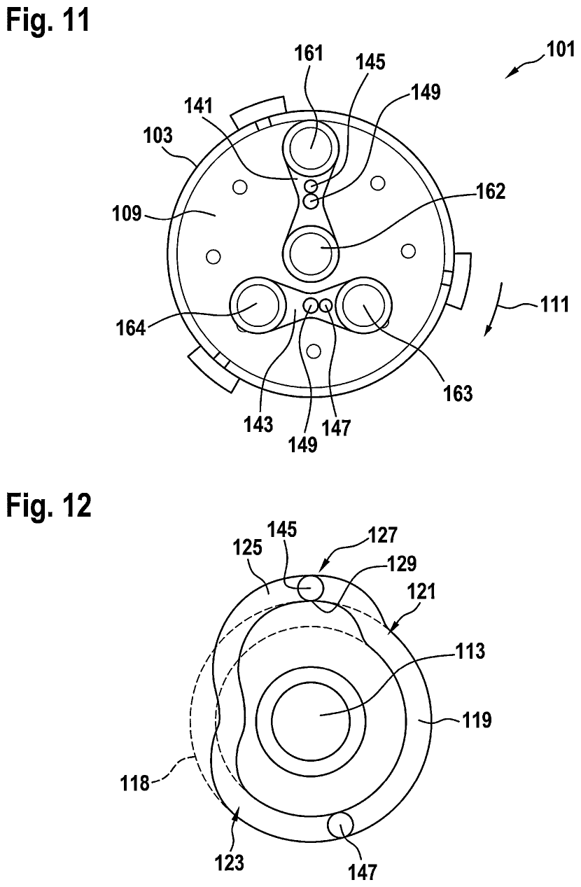

FIG. 11 is a schematic representation of the filter changer with a pivoted-in filter pivot arm and a filter in an optical passage.

FIG. 12 is a schematic representation of the guide groove corresponding to FIG. 11.

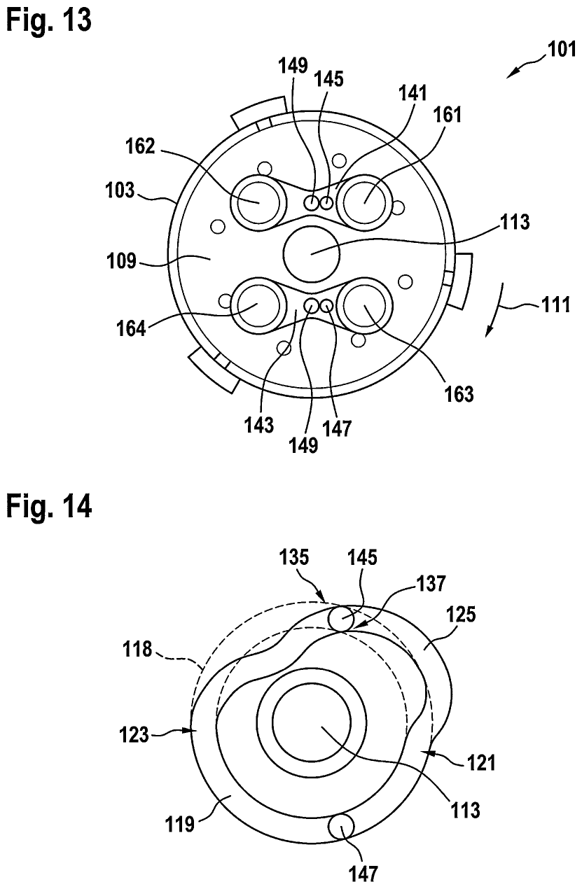

FIG. 13 is a schematic representation of the filter changer with both filter pivot arms in the pivoted-out state.

FIG. 14 is a schematic representation of the guide groove corresponding to FIG. 13.

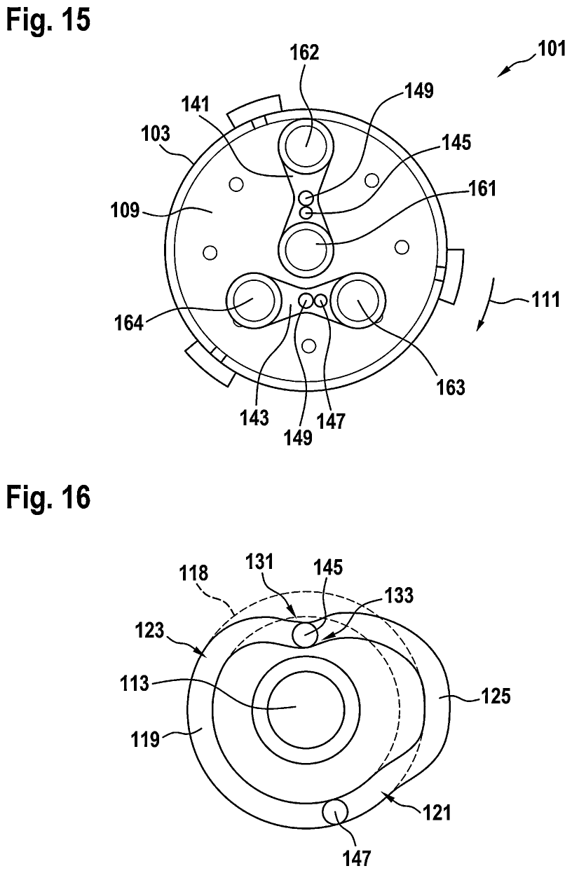

FIG. 15 is a schematic representation of the filter changer with the again pivoted-in filter pivot arm with the other filter in the optical passage.

FIG. 16 is a schematic representation of the guide groove corresponding to FIG. 15.

An endoscope system 171 has a camera head 177, a filter changer 101 and an endoscope 173. The filter changer 101 is connected to the camera head 177 by means of a camera receptacle 179 and to the endoscope 173 by means of an endoscope receptacle 175 (FIGS. 1 and 2). Furthermore, the filter changer 101 has a housing 103 with a base plate 107 designed as a proximal cover. The base plate 107 is externally connected to a distal cover 108 by means of a connector 105, which may be a screw, rivet, pin, etc.

A rotatable filter wheel 109 is arranged between the distal cover 108 and the base plate 107. The filter wheel 109 may be rotatable by means of a motor (not shown). The base plate 107 and the filter wheel 109 each have an optical passage 113 around an optical axis 115. The rotatable filter wheel 109 has a guide groove 117 with a concentric guide track portion 119 and a non-concentric guide track portion 125. The circumferential guide groove 117 is arranged between the optical passage 113 and an outer diameter of the filter wheel 109. Eight pressure piece receptacles 167 are evenly distributed around the outside of the guide groove 117 (see FIG. 7).

The base plate 107 is stationary and, on its side facing the filter wheel 109, has a pressure piece 165, which can be partially received in one of the pressure piece receptacles 167. Furthermore, a first filter pivot arm 141 and a second filter pivot arm 143 are each pivotably fastened by means of a rotation axle 149 to this side of the base plate 107. The first filter pivot arm 141 and the second filter pivot arm 143 are fastened by means of the particular rotation axles 149 to the base plate 107 in such a way that the first guide pin 145 of the first filter pivot arm 141 and the second guide pin 147 of the second filter pivot arm 143 are arranged in the guide groove 117. FIG. 6 shows an alignment of these two filter pivot arms 141, 143 in an initial position, in which both filter pivot arms 141, 143 are pivoted out of the optical passage 113 and arranged in parallel with one another.

The filter pivot arms 141, 143 may be double-lobe-shaped with two opposite ends. The first filter pivot arm 141 has a rotation axle 149 and a first guide pin 145 and, at its lobe-shaped ends, a first filter receptacle 151 with a received first filter 161 on the one side and, on the opposite side, a second filter receptacle 152 with a second filter 162 held therein. As shown by way of example for the second filter pivot arm 143 in FIG. 5, the double-lobe-shaped filter pivot arm has a continuous central bore 148 for receiving the rotation axle 149. A second guide pin 147 is arranged next to this bore 148. The lobe-shaped end of the filter holder 143 next to the second guide pin 147 has a third filter receptacle 153 with a third filter 163, while a fourth filter receptacle 154 with a fourth filter 164 is arranged at the opposite second lobe-shaped end.

The detailed design and the course of the guide groove 117 in the filter wheel 109 is shown in FIG. 8. The concentric guide track portion 119 with a circumference of 180° has a beginning 121 and an opposite end 123 for a clockwise rotation direction 111 (for an opposite, counterclockwise rotation, the beginning 121 and the end 123 are correspondingly reversed). This concentric guide track portion 119 has a constant radius 118 to the optical axis 115. At the end 123, the concentric guide track portion 119 transitions into the non-concentric guide track portion 125. Likewise, the beginning 121 of the concentric guide track portion 119 transitions into an opposite end of the non-concentric guide track portion 125.

The non-concentric guide track portion 125 has, starting from the beginning 121 of the concentric guide track portion 119 and thus in the direction opposite the rotation direction 111, a first subportion 127 with a guide groove position 129 at a maximum distance. This first subportion 127 is designed as an outward-bent circular arc portion with a central angle between the non-concentric guide track portion 125 and the optical passage 113. The guide groove position 129 at a maximum distance has a greater distance from the optical axis 115 than the radius 118 of the concentric guide track portion 119. In the direction opposite the rotation direction 111, the first subportion 127 is adjoined by a transition portion 135, which has a larger circular arc than the first subportion 127. The transition portion 135 has a transition position 137, at which the transition portion 135 intersects the radius 118 of the concentric guide track portion 119. Thereafter, in the direction opposite the rotation direction 111, the transition portion 135 transitions into a second subportion 131. The second subportion 131 is designed as a shorter inward-directed circular arc with a central angle outside the guide groove 117 directed toward the outer circumference of the filter wheel 109. The second subportion 131 has a guide groove position 133 at a minimum distance, at which the guide groove 117 and the non-concentric guide track portion 125 are at the smallest distance to the optical axis 115. This distance is approximately 50% of the radius 118. Subsequently, in the direction opposite the rotation direction 111, the second subportion 131 again transitions in an outward-directed circular arc, tapering to the radius 118, into the end 123 of the concentric guide track portion 119.

The following operations are carried out by means of the filter changer 101 and the endoscope system 171.

Starting from an initial position of the first filter pivot arm 141 and the second filter pivot arm 143, these two filter pivot arms 141, 143, each in a pivoted-out state, are parallel to one another and the optical passage 113 is freely passable (see FIG. 9 and the initial positions shown in FIGS. 3, 4 and 6). Here, the first guide pin 145 of the first filter pivot arm 141 is arranged at the beginning 121 of the concentric guide track portion 119 and the second guide pin 147 of the second filter pivot arm 143 is located in the concentric guide track portion 119 shortly before the end 123 thereof. Both guide pins 145 and 147 thus lie on the radius 118 and, accordingly, the two filter pivot arms 141, 143 are not pivoted into the optical passage 113, but are in the initial position (FIGS. 9 and 10).

When the filter wheel 109 is rotated manually by an operator or by means of a motor (not shown) in the rotation direction 111, the first guide pin 145 is pushed from the beginning 121 of the concentric guide track portion 119 into the first subportion 127 of the non-concentric guide track portion 125 until it reaches the guide groove position 129 at a maximum distance. Due to the forces acting on the first guide pin 145 through the inner walls of the guide groove 117 in this first subportion 127 and due to the pivotable mounting of the first filter pivot arm 141 by means of the rotation axle 149 on the stationary base plate 107, the first filter pivot arm 141 rotates clockwise, wherein the first guide pin 145 is pressed perpendicularly over the rotation axle 149 of the first filter pivot arm 141 and the second filter 162 of the first filter pivot arm 141 is thereby pivoted into the optical passage 113 (FIGS. 11 and 12). This pivoted-in state is indicated to the user haptically by the pressure piece 165 engaging in the corresponding pressure piece receptacle 167.

Meanwhile, the second guide pin 147 of the second filter pivot arm 143 is located in the concentric guide track portion 119 and has moved in the direction opposite the rotation direction 111, in the direction of the beginning 121 of the concentric guide track portion 119. The second filter pivot arm 143 thus remains in the initial position and is not pivoted into the optical passage 113, since the second guide pin 147 is still located to the right of the rotation axle 149 (FIG. 11).

After the user has used the second filter 162 in the beam path, the user rotates the filter wheel 109 further in the rotation direction 111. As a result, the first guide pin 145 is pushed in the direction opposite the rotation direction 111 from the first subportion 127 into the transition portion 135 and there reaches the transition position 137 in which the first guide pin 145 is on the radius 118. The second guide pin 147 is simultaneously pushed in the concentric guide track portion 119 further in the direction opposite the rotation direction 111, in the direction of the beginning 121. Since both guide pins 145, 147 are now on the radius 118, the first filter pivot arm 141 and the second filter pivot arm 143 are again in the initial position and are pivoted out and arranged in parallel with one another (FIGS. 13 and 14).

In order now to pivot-in the first filter 161 of the first filter pivot arm 141 into the optical passage 113, the user subsequently rotates the filter wheel 109 further in the rotation direction 111, whereby the corresponding movement of the guide groove 117 moves the first guide pin 145 of the first filter pivot arm 141 from the transition position 137 into the second subportion 131 of the non-concentric guide track portion 125 until the first guide pin 145 reaches the guide groove position 133 at a minimum distance. In the process, the first guide pin 145 is pressed below the rotation axle 149 so that the first filter pivot arm 141 has accordingly rotated counterclockwise and the first filter 161 is pivoted into the optical passage 113. The pivoting-in of the first filter 161 is again indicated to the user by the pressure piece 165 engaging in the corresponding pressure piece receptacle 167. At the same time, during the rotation of the filter wheel 109, the second guide pin 147 is moved further in the direction of the beginning 121 of the concentric guide track portion 119. Since the second guide pin 147 is still on the radius 118 of the concentric guide track portion 119, the second filter pivot arm 143 remains in the initial position (FIGS. 15 and 16).

If the user now rotates the filter wheel 109 further in the rotation direction 111, the second guide pin 147 reaches the beginning 121 of the concentric guide track portion 119 and, during further rotation in the rotation direction 111, successively assumes the successive guide groove positions in the non-concentric guide track portion 125 that were described above for the first guide pin 145, while the first pin 145 is accordingly on the radius 118 within the concentric guide track portion 119 and consequently remains pivoted out.

Optionally, the user can also instead rotate the filter wheel 109 in the direction opposite the rotation direction 111, in which case the course of the first guide pin 145 in the non-concentric guide track portion 125 is traversed in the opposite direction to the description above and the first filter pivot arm 141 performs the corresponding pivoting movements in the opposite order to the description above, while the second guide pin 147 again remains on the radius 118 in the concentric guide track portion 119. Likewise, the user can of course change from the clockwise rotation direction 111 to a counterclockwise rotation direction at any position of the guide pins 145, 147 in the guide groove 117.

This provides a filter changer 101 by means of which changing between different filters 161, 162, 163, 164 can take place quickly, efficiently and precisely and which can be used with different types of endoscopes 173 and camera heads 177.

Below are described some of the potential variations of the embodiments disclosed herein.

A filter changing apparatus (101) for an endoscopic camera, wherein the filter changing apparatus (101) has a base plate (107), a rotatable filter plate (109), an optical passage (113) with an optical axis (115) and at least two pivotable filter holders (141, 143), each with at least one receptacle (151, 152, 153, 154) for an optical filter (161, 162, 163, 163), wherein the rotatable filter plate (109) has a guide groove (117) with a circumferential guide track and is rotatable relative to the base plate (107), the filter holders (141, 143) are each arranged rotatably on the base plate (107) by means of a rotation axle (149) and each have a guide element (145, 147), wherein each guide element (145, 147) is at least partially arranged in the guide groove (117), characterized in that the guide groove (117) has a first portion (119), concentric with the optical axis (115), and a second portion (125), non-concentric with the optical axis (115), so that, when at least one guide element (145, 147) is arranged in the concentric portion (119), the particular filter holder (141, 143) is arranged in an initial position, free of the optical passage (113), and/or, when at least one guide element (145, 147) is arranged in the second, non-concentric portion (125), the associated filter holder (141, 143) can be pivoted into the optical passage (113).

The filter changing apparatus (101) described above, characterized in that the first, concentric portion (119) of the guide groove (117) has a length with an angular width, with the optical axis (115) as the angle vertex, in a range of 170° to 190°, in particular of 175° to 185°, preferably of 178° to 182°.

A filter changing apparatus described above, characterized in that the first, concentric portion (119) of the guide groove (117) has a starting position at its one end (121) and an end position at its other end (123) so that, when the guide element (145) of the first filter holder (141) is arranged in the starting position and the guide element (147) of the second filter holder (143) is arranged in the end position, both filter holders (141, 143) are in a position, free of the optical passage (113), and/or in the particular initial position.

A filter changing apparatus (101) described above, characterized in that the rotation axle (149) of each filter holder (141, 143) is arranged between the optical axis (115) and the guide groove (117) or between the guide groove (117) and an outer diameter of the rotatable filter plate (109).

A filter changing apparatus (101) described above, characterized in that the filter holder(s) (141, 143) have a receptacle (151, 152, 153, 154) at each of two opposite ends and the rotation axle (149) is arranged centrally between the two opposite ends.

A filter changing apparatus (101) described above, characterized in that the guide element (145, 147) of each filter holder (141, 143) is arranged between the rotation axle (149) and one of the two receptacles (151, 152, 153, 154), in particular closer to the rotation axle (149) than to the particular receptacle (151, 152, 153, 154).

A filter changing apparatus (101) described above, characterized in that the second, non-concentric portion (125) of the guide groove (117) has a first subportion (127) with a guide groove position (129) at a maximum distance, which has a greater distance to the optical axis (115) than the first, concentric portion (119) so that, when the particular guide element (145, 147) is arranged at the guide groove position (129) at a maximum distance, the particular filter holder (141, 143) is pivoted into the optical passage (113).

A filter changing apparatus (101) described above, characterized in that the second, non-concentric portion (125) of the guide groove (117) has a second subportion (131) with a guide groove position (133) at a minimum distance, which subportion has a smaller distance to the optical axis (115) than the first, concentric portion (119) so that, when the particular guide element (145, 147) is arranged at the guide groove position (133) at a minimum distance, the receptacle (151, 152, 153, 154) of the particular filter holder (141, 143) that is arranged closest to the guide element (145, 147) is pivoted into the optical passage (113).

A filter changing apparatus (101) described above, characterized in that a transition portion (135) with a transition position (137) is arranged between the first subportion (127) and the second subportion (131) of the second, non-concentric portion (125) so that, when the particular guide element (145, 147) is arranged, both receptacles (151, 152, 153, 154) of the particular filter holder (141, 143) are pivoted out of the optical passage (113).

A filter changing apparatus (101) described above, characterized in that the transition position (137) and the first, concentric portion (119) have the same distance to the optical axis (115) so that, when the particular guide element (145, 147) is moved along the guide groove (117), the particular filter holder (141, 143) can be pivoted out directly into a position free of the optical passage (113) and/or into the initial position after being arranged in the optical passage (113).

A filter changing apparatus (101) described above, characterized in that the filter holder (141, 143) is or the filter holders (141, 143) are dumbbell-shaped or double-lobe-shaped.

A filter changing apparatus (101) described above, characterized in that the filter changing apparatus (101) has a feedback device (165, 167) when a guide groove position, pivoted-in position and/or pivoted-out position is reached.

A filter changing apparatus (101) described above, characterized in that the first, concentric portion (119) and the second, non-concentric portion (125) are designed in such a way that the filter holders (141, 143) have opposite directions of rotation about the particular rotation axle (14) and/or have overlapping rotation radii.

A camera head (177) for an endoscope (173), wherein the camera head (177) has an image sensor, an opening for receiving light of an image along an optical path and an optical lens system for focusing the light on the image sensor, characterized in that the camera head (177) has at least one filter changing apparatus (101) described above.

A retrofit kit for retrofitting a camera head and/or an endoscope, characterized in that the retrofit kit has at least one filter changing apparatus (101) described above such that the filter changing apparatus (101) can be arranged between a proximal end of the endoscope (173) and a distal end of the camera head (177).

The above-described exemplary embodiments are intended to illustrate the principles of the disclosed technology, but not to limit the scope of the disclosed technology. Various other embodiments and modifications to these exemplary, non-exhaustive embodiments may be made by those skilled in the art without departing from the scope of the disclosed technology. For example, in some instances, one or more features disclosed in connection with one embodiment can be used alone or in combination with one or more features of one or more other embodiments. More generally, the various features described herein may be used in any working combination.

### LIST OF REFERENCE SIGNS

101 filter changer
103 housing
105 connector
107 base plate/proximal cover
108 distal cover
109 filter wheel
111 rotation direction
113 optical passage
115 optical axis
117 guide groove
118 radius
119 concentric guide track portion
121 beginning of the concentric guide track portion
123 end of the concentric guide track portion
125 non-concentric guide track portion
127 first subportion
129 guide groove position at a maximum distance
131 second subportion
133 guide groove position at a minimum distance
135 transition portion
137 transition position
141 first filter pivot arm
143 second filter pivot arm
145 first guide pin
147 second guide pin
148 bore
149 rotation axle
151 first filter receptacle
152 second filter receptacle
153 third filter receptacle
154 fourth filter receptacle
161 first filter
162 second filter
163 third filter
164 fourth filter
165 pressure piece
167 pressure piece receptacle
171 endoscope system
173 endoscope
175 endoscope receptacle
177 camera head
179 camera receptacle

1. A filter changing apparatus for an endoscopic camera, wherein the filter changing apparatus has a base plate, a rotatable filter plate, an optical passage with an optical axis and at least two pivotable filter holders, each with at least one receptacle for an optical filter, wherein the rotatable filter plate has a guide groove with a circumferential guide track and is rotatable relative to the base plate, the filter holders are each arranged rotatably on the base plate by means of a rotation axle and each have a guide element, wherein each guide element is at least partially arranged in the guide groove, characterized in that the guide groove has a first portion, concentric with the optical axis, and a second portion, non-concentric with the optical axis, so that, when at least one guide element is arranged in the concentric portion, the particular filter holder is arranged in an initial position, free of the optical passage, and/or, when at least one guide element is arranged in the second, non-concentric portion, the associated filter holder can be pivoted into the optical passage.

2. The filter changing apparatus of claim 1, wherein the first, concentric portion of the guide groove has a length with an angular width, with the optical axis as the angle vertex, in a range of 170° to 190°.

3. The filter changing apparatus according to claim 1, wherein the first, concentric portion of the guide groove has a starting position at its one end and an end position at its other end such that, when the guide element of the first filter holder is arranged in the starting position and the guide element of the second filter holder is arranged in the end position, both filter holders are in a position, free of the optical passage, and in the initial position.

4. The filter changing apparatus of claim 1, wherein the rotation axle of each of the filter holders is arranged between the optical axis and the guide groove or between the guide groove and an outer diameter of the rotatable filter plate.

5. The filter changing apparatus of claim 1, wherein the filter holders have a receptacle at each of two opposite ends and the rotation axle is arranged centrally between the two opposite ends.

6. The filter changing apparatus of claim 5, wherein the guide element of each filter holder is arranged between the rotation axle and one of the two receptacles.

7. The filter changing apparatus of claim 6, wherein the guide element is arranged closer to the rotation axle than to the respective receptacle.

8. The filter changing apparatus of claim 1, wherein the second, non-concentric portion of the guide groove has a first subportion with a guide groove position at a maximum distance, which has a greater distance to the optical axis than the first, concentric portion such that, when the respective guide element is arranged at the guide groove position at a maximum distance, the respective filter holder is pivoted into the optical passage.

9. The filter changing apparatus of claim 1, wherein the second, non-concentric portion of the guide groove has a second subportion with a guide groove position at a minimum distance, which subportion has a smaller distance to the optical axis than the first, concentric portion such that, when the respective guide element is arranged at the guide groove position at a minimum distance, the receptacle of the respective filter holder that is arranged closest to the guide element is pivoted into the optical passage (113).

10. The filter changing apparatus of claim 1, wherein a transition portion with a transition position is arranged between the first subportion and the second subportion of the second, non-concentric portion such that, when the respective guide element is arranged, both receptacles of the respective filter holder are pivoted out of the optical passage.

11. The filter changing apparatus of claim 10, wherein the transition position and the first, concentric portion have the same distance to the optical axis such that, when the respective guide element is moved along the guide groove, the respective filter holder can be pivoted out directly into a position free of the optical passage and into the initial position after being arranged in the optical passage.

12. The filter changing apparatus of claim 1, wherein the filter holder is, or the filter holders are, dumbbell-shaped or double-lobe-shaped.

13. The filter changing apparatus of claim 1, wherein the filter changing apparatus has a feedback device when a guide groove position, pivoted-in position, or pivoted-out position is reached.

14. The filter changing apparatus of claim 1, wherein the first, concentric portion and the second, non-concentric portion are configured in such a way that the filter holders have opposite directions of rotation about their respective rotation axle or have overlapping rotation radii.

15. A camera head for an endoscope, wherein the camera head includes an image sensor, an opening for receiving light of an image along an optical path and an optical lens system for focusing the light on the image sensor, wherein in that the camera head further includes at least one filter changing apparatus, the filter changing apparatus including a base plate, a rotatable filter plate, an optical passage with an optical axis and at least two pivotable filter holders, each with at least one receptacle for an optical filter, wherein the rotatable filter plate has a guide groove with a circumferential guide track and is rotatable relative to the base plate, the filter holders are each arranged rotatably on the base plate by means of a rotation axle and each have a guide element, wherein each guide element is at least partially arranged in the guide groove, characterized in that the guide groove has a first portion, concentric with the optical axis, and a second portion, non-concentric with the optical axis, so that, when at least one guide element is arranged in the concentric portion, the particular filter holder is arranged in an initial position, free of the optical passage, and/or, when at least one guide element is arranged in the second, non-concentric portion, the associated filter holder can be pivoted into the optical passage.

16. The camera head of claim 15, wherein the rotation axle of each of the filter holders is arranged between the optical axis and the guide groove or between the guide groove and an outer diameter of the rotatable filter plate.

17. The camera head of claim 15, wherein the second, non-concentric portion of the guide groove has a first subportion with a guide groove position at a maximum distance, which has a greater distance to the optical axis than the first, concentric portion such that, when the respective guide element is arranged at the guide groove position at a maximum distance, the respective filter holder is pivoted into the optical passage.

18. A retrofit kit for retrofitting a camera head or an endoscope, characterized in that the retrofit kit has at least one filter changing apparatus that can be arranged between a proximal end of the endoscope and a distal end of the camera head, wherein the at least one filter changing apparatus includes a base plate, a rotatable filter plate, an optical passage with an optical axis and at least two pivotable filter holders, each with at least one receptacle for an optical filter, wherein the rotatable filter plate has a guide groove with a circumferential guide track and is rotatable relative to the base plate, the filter holders are each arranged rotatably on the base plate by means of a rotation axle and each have a guide element, wherein each guide element is at least partially arranged in the guide groove, characterized in that the guide groove has a first portion, concentric with the optical axis, and a second portion, non-concentric with the optical axis, so that, when at least one guide element is arranged in the concentric portion, the particular filter holder is arranged in an initial position, free of the optical passage, and/or, when at least one guide element is arranged in the second, non-concentric portion, the associated filter holder can be pivoted into the optical passage.

19. The retrofit kit of claim 18, wherein the rotation axle of each of the filter holders is arranged between the optical axis and the guide groove or between the guide groove and an outer diameter of the rotatable filter plate.

20. The retrofit kit of claim 19, wherein the second, non-concentric portion of the guide groove has a first subportion with a guide groove position at a maximum distance, which has a greater distance to the optical axis than the first, concentric portion such that, when the respective guide element is arranged at the guide groove position at a maximum distance, the respective filter holder is pivoted into the optical passage.
